(19) United States
(12) Patent Application Publication
(10) Pub. No.: US 2009/0272025 A1
(43) Pub. Date: Nov. 5, 2009

(54) Disinfectant household bomb

(76) Inventor: SUSAN Marie Wolosuk, Yale, VA (US)

Correspondence Address: JAMES RAY & ASSOCIATES, 2640 PITCAIRN ROAD, MONROEVILLE, PA, 15146, US

(21) Appl. No.: 12/150,795

(22) Filed: May 1, 2008

(51) Int. Cl. A01M 13/00

(52) U.S. Cl. 43/129; 43/1

(57) ABSTRACT

An apparatus for killing germs and bacteria in an enclosed area such as a room and an office. The apparatus comprises a fogger member having each of a predetermined shape and a predetermined size and formed of a predetermined material. There is an antibacterial agent disposed within the fogger member for killing bacteria on contact and a germicide is also disposed in the fogger member for killing germs on contact. A pull tab is disposed on a top portion of the fogger member for releasing such antibacterial agent and such germicide in a form of a fog.

### FIELD OF THE INVENTION

The present invention relates, in general, to a household disinfectant, and, more particularly, the present invention relates to a bomb fogger containing an anti-bacterial agent and a germicide for killing germs and bacteria.

### BACKGROUND OF THE INVENTION

Many homes, business offices, medical facilities and classrooms are full of germs and bacterial that can be spread through simple physical contact or picked up through contaminate air. Cleaning the rooms can limit the spread of germs but eliminating the risk of catching germs is difficult without effective, all-over cleaning.

Thus, it would be advantageous if there were a simple and effective means for sanitizing a room, including the air, to eliminate harmful germs and bacteria that leads to colds and flu.

### SUMMARY OF THE INVENTION

In a first aspect the present invention provides an apparatus for killing germs and bacteria in an enclosed area such as a room and an office. The apparatus comprises a fogger member having each of a predetermined shape and a predetermined size and formed of a predetermined material. There is an antibacterial agent disposed within the fogger member for killing bacteria on contact and a germicide is also disposed in the fogger member for killing germs on contact. A means is disposed on a top portion of the fogger member for releasing such antibacterial agent and such germicide in a form of a fog.

In a second aspect there is provided a method for sanitizing an enclosed area such as a room and an office. The method comprises the steps of selecting an appropriate sized fogger member having a predetermined shape and containing an antibacterial agent and a germicide and a step of dispensing such antibacterial agent and such germicide in said fogger member in a form of a fog in an enclosed area. There is a step of evacuating such enclosed area for a predetermined time and a step of permitting such antibacterial agent and such germicide to work for the predetermined time and a step of opening the enclosed area after such predetermined time has elapsed.

### OBJECTS OF THE INVENTION

It is, therefore, one of the primary objects of the present invention to provide a household fogger designed to kill germs and bacteria in a room and all its exposed areas.

Another object of the present invention is to provide a household fogger that is under pressure.

Still another object of the present invention is to provide a household fogger that gently releases the antibacterial agent and the germicide in the form of a fog.

Yet another object of the present invention is to provide a a household fogger that has an easy release top.

In addition to the various objects and advantages of the invention which have been described in some specific detail above it should be noted that various other objects and advantages of the present invention will become more readily apparent to those persons who are skilled in the relevant art from the following more detailed description, particularly when such description is taken in conjunction with the appended claims.

### BRIEF DESCRIPTION OF THE DRAWINGS

FIG. 1 is a side view of the apparatus according to an embodiment of the invention.

FIG. 2 is a perspective view of the apparatus showing two different size foggers.

FIG. 3 is a is a perspective view of the apparatus shown in FIG. 1 showing the pull tab for releasing the disinfectant.

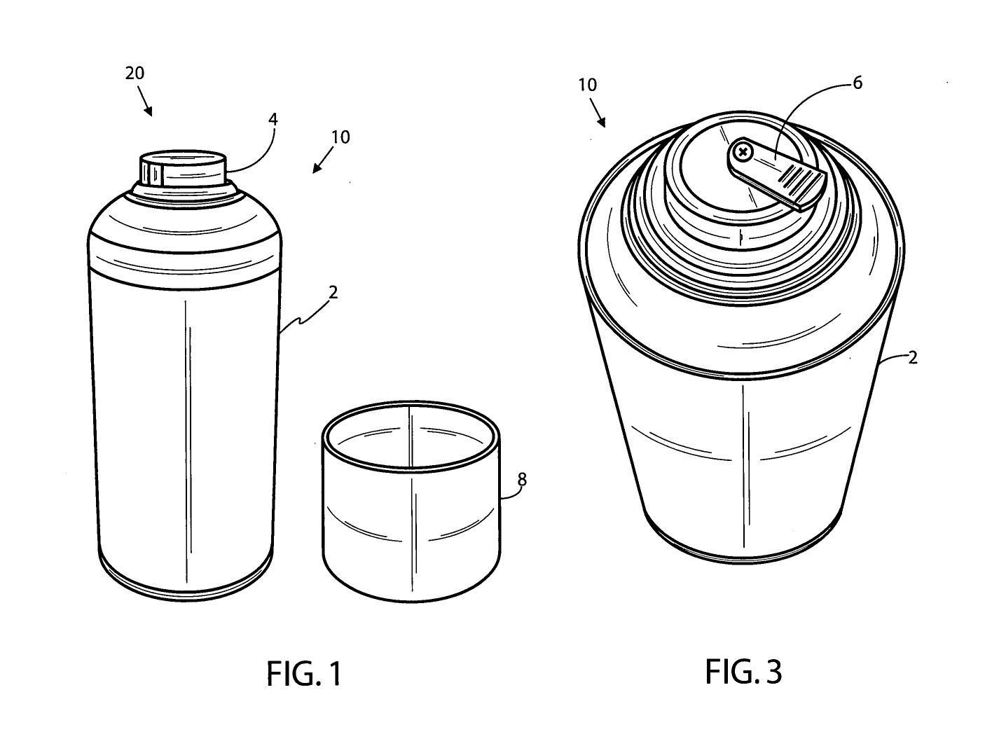

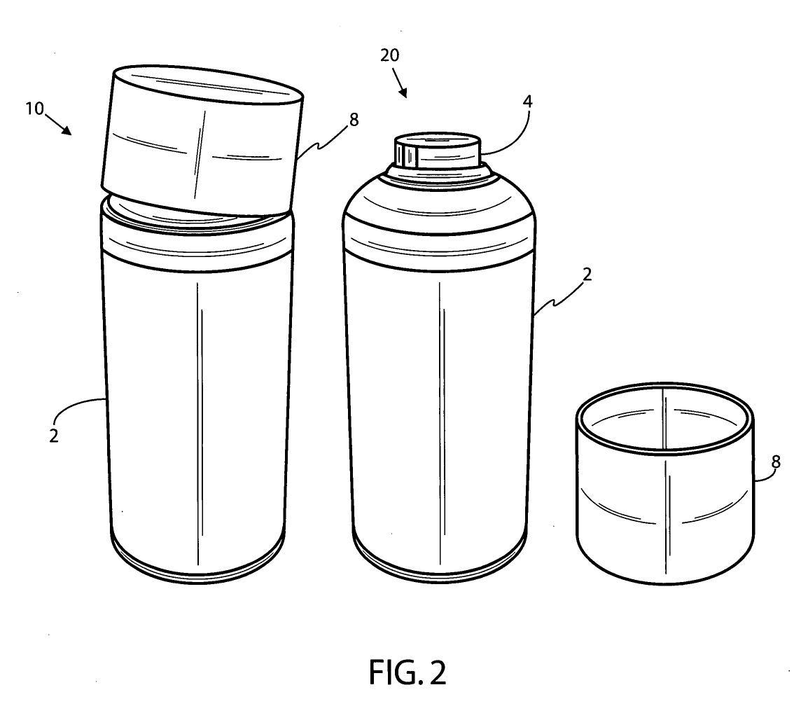

### BRIEF DESCRIPTION OF THE PRESENTLY PREFERRED AND VARIOUS ALTERNATIVE EMBODIMENTS OF THE INVENTION

Prior to proceeding with the more detailed description of the present invention it should be noted that, for the sake of clarity, identical components which have identical functions have been designated by identical reference numerals throughout the several views illustrated in the drawings.

In a first aspect the present invention provides an apparatus, generally designated 10, for killing germs and bacteria in an enclosed area such as a room and an office. The apparatus 10 comprises a fogger member 2 having each of a predetermined shape and a predetermined size and formed of a predetermined material. There is an antibacterial agent disposed within the fogger member 2 for killing bacteria on contact and a germicide is also disposed in the fogger member 2 for killing germs on contact. A means, generally designated 20, is disposed on a top portion 4 of the fogger member 2 for releasing such antibacterial agent and such germicide in a form of a fog.

Although the shape of the fogger member could any of a variety of shapes it is presently preferred that such shape is substantially cylindrical. Such predetermined material for forming the fogger member is selected from one of plastic and metal and combinations thereof. It is presently preferred that such material is metal.

The predetermined size of fogger member 2 is one of different sizes. There is one size for small rooms and a larger size for larger rooms. If a room is even larger then it would be necessary to use two or more fogger members 2. Such predetermined size for use in a large room is between about 5¾ inches and about 6 inches tall. The diameter of the cylindrical fogger member 2 is between about 2½ inches and about 2¾ inches. The size of the smaller fogger member for use with small rooms or offices is between about 2¾ inches and about 3¼ inches tall while the diameter is between about 1⅝ and about 2 inches.

Such means 20 for releasing such antibacterial agent and such germicide in a form of a fog includes a pull tab 6. The fogger member 2 also includes a cap member 8 for covering a top of the fogger member 2.

In a second aspect there is provided a method for sanitizing an enclosed area such as a room and an office. The method comprises the steps of selecting an appropriate sized fogger member 2 having a predetermined shape and containing an antibacterial agent and a germicide and a step of dispensing such antibacterial agent and such germicide in said fogger member 2 in a form of a fog in an enclosed area. There is a step of evacuating such enclosed area for a predetermined time and a step of permitting such antibacterial agent and such germicide to work for the predetermined time and a step of opening the enclosed area after such predetermined time has elapsed. It should be noted that the predetermined time is at least one hour.

Such step of dispensing such antibacterial agent and such germicide further includes the step of pulling a pull tab 6 on the top of the fogger member 2 to dispense such antibacterial agent and such germicide. Such method also includes the step of using a predetermined number of fogger members for larger rooms.

Thus, the present invention provides a household and office antibacterial and anti-germicidal fogger that is designed to kill germs and bacteria in a room and all its exposed areas, preventing the spread of colds and flu. The canned antibacterial agent and germicide work in a manner similar to canned bug foggers in which the canned agents are dispensed through a fog, which is intended to kill bugs. The present invention provides a canned container containing such antibacterial agent and germicide under pressure. The agents are gently released in the form of a fog through an easy release top. The unit is made for use while the home or office is vacant. The user sets up the appropriate size can, allowing the fog to work for a predetermined amount of time. The room or office should be vacant for at least one hour for the fog to work.

The fogger member comes in two different sizes. A large can which is substantially 6 inches tall is designed for use in a large room or office, while a small can, substantially three inches tall will conveniently sanitize a small room or a single office. Depending on the room size, larger rooms may require more than one fogger to substantially sanitize the area.

While a presently preferred embodiment and alternate embodiments of the present invention have been described in detail above, it should be understood that various other adaptations and/or modifications of the invention can be made by those persons who are particularly skilled in the art without departing from either the spirit of the invention or the scope of the appended claims.

I claim:

1. An apparatus for killing germs and bacteria in an enclosed area such as a room and an office, said apparatus comprising:
(a) a fogger member having each of a predetermined shape and a predetermined size and formed of a predetermined material;
(b) an antibacterial agent disposed within said fogger member for killing bacteria on contact;
(c) a germicide disposed in said fogger member for killing germs on contact; and
(d) a means disposed on a top portion of said fogger member for releasing said antibacterial agent and said germicide in a form of a fog.

2. The apparatus, according to claim 1, wherein said predetermined shape of said fogging member is substantially cylindrical.

3. The apparatus, according to claim 1, wherein said means disposed on a top portion of said apparatus includes a pull tab for releasing said antibacterial agent and said germicide.

4. The apparatus, according to claim 1, wherein said predetermined material for forming said fogger member is selected from one of plastic and metal.

5. The apparatus, according to claim 4, wherein said predetermined material is metal.

6. The apparatus, according to claim 1, wherein said predetermined size of said fogger member for fogging a large room is between about 5¾ inches and about 6 inches tall.

7. The apparatus, according to claim 1, wherein said predetermined size of said fogger member for fogging a small room is between about 2¾ inches and about 3¼ inches tall.

8. The apparatus, according to claim 1, wherein said fogger member further includes a cap member for covering a top of said fogger member.

9. A method for sanitizing an enclosed area such as a room and an office, said method comprising the steps of:
(a) selecting an appropriate sized fogger member having a predetermined shape and containing an antibacterial agent and a germicide;
(b) dispensing said antibacterial agent and said germicide in said fogger member in a form of a fog in an enclosed area;
(c) evacuating said enclosed area for a predetermined time;
(d) permitting said antibacterial agent and said germicide to work for said predetermined time; and
(e) opening said enclosed area after said predetermined time.

10. The method, according to claim 9, wherein said predetermined time is at least one hour.

11. The method, according to claim 9, wherein said step (b) of said method further includes the step of pulling a tab on a top of said fogger member to dispense said antibacterial agent and said germicide.

12. The method, according to claim 12, wherein said method includes a step of using a predetermined number of fogger members for larger rooms.
